(19) United States
(12) Patent Application Publication
(10) Pub. No.: US 2011/0106157 A1
(43) Pub. Date: May 5, 2011

(54) Self-Locking Interference Bone Screw for use with Spinal Implant

(75) Inventors: Anthony J. Melkent, Memphis, TN (US); Thomas A. Carls, Memphis, TN (US); Lindsey G. Waugh, Memphis, TN (US)

(73) Assignee: WARSAW ORTHROPEDIC, INC., Warsaw, IN (US)

(21) Appl. No.: 12/609,520

(22) Filed: Oct. 30, 2009

(51) Int. Cl. A61B 17/86; A61B 17/70

(52) U.S. Cl. 606/246; 606/309; 606/312

(57) ABSTRACT

The present invention is generally directed to a unique bone screw which, in one form, may be used to engage with and attach an implant to bone or bony tissue. In one embodiment, a bone screw includes an elongate shank extending along a longitudinal axis from an enlarged proximal head portion to a distal tip. The elongate shank includes a first portion having a first non-tapered, linear external profile and a second portion having a first tapered external profile that increases toward the proximal head portion. The second portion is positioned between the first portion and the head portion and terminates distally of the proximal head portion. In one form of this embodiment, the bone screw can be advanced through a passage of an implant such that an interference fit is provided between the bone screw and the implant.

### BACKGROUND

The present invention generally relates to bone screws that are used to attach one or more implants to bone or bony tissue, and more particularly relates to a self-locking interference bone screw.

Various types of fasteners are used to engage implants or other devices to bone. In the spinal field, bone screws are commonly used to attach plates, rods and/or other types of implants or devices to one or more vertebrae. In some instances, bone screws can become unthreaded or disengaged following attachment of an implant to bone or one or more vertebrae. This unthreading or disengagement can result in failed surgical procedures and/or interference with patient anatomy surrounding the implantation site, which can in turn result in trauma to adjacent tissue and patient discomfort and can sometimes present a need for additional surgical procedures.

Thus, there remains a need for improved bone screws and methods for using the same.

### SUMMARY

One non-limiting embodiment of the present invention is directed to a bone screw structured to engage with an implant such as, for example, a bone plate, rod, or interbody stabilization device. In a more particular form, the bone screw is configured to engage with a passage in the implant such that an interference fit is provided between the bone screw and the implant, thereby reducing the likelihood of the bone screw becoming disengaged from the implant and/or bone. However, in other embodiments, different forms and applications of the bone screw are envisioned.

Another embodiment of the present invention is directed to a bone screw that includes an elongate shank extending along a longitudinal axis from an enlarged proximal head portion to a distal tip. The elongate shank includes a first portion having a first non-tapered, linear external profile and a second portion having a first tapered external profile that increases toward the proximal head portion. The second portion is positioned between the first portion and the head portion, and terminates distally of the proximal head portion.

In another embodiment, a stabilization system includes a bone screw and an implant including at least one passage extending therethrough. The bone screw includes an elongate shank extending along a longitudinal axis from an enlarged proximal head portion to a distal tip. The elongate shank includes a first portion having a first non-tapered, linear external profile and a second portion having a first tapered external profile that increases toward the proximal head portion. The second portion is positioned between the first portion and the head portion, and terminates distally of the proximal head portion. The passage of the implant is configured to engage with at least the second portion of the elongate shank of the bone screw to provide an interference fit between the bone screw and the implant.

In yet another embodiment, a bone screw includes an elongate shank extending along a longitudinal axis from an enlarged proximal head portion to a distal tip. The elongate shank includes a first threaded portion and a second portion positioned adjacent to and proximal of the first threaded portion. The second portion defines a distal facing annular shoulder. In one form of this embodiment, the annular shoulder extends radially beyond an external profile of the first threaded portion. In another form of this embodiment, the second portion is also threaded.

Other embodiments include unique methods, systems, devices, kits, assemblies, equipment, and/or apparatus involving a bone screw and/or an implant.

Further embodiments, forms, features, aspects, benefits, objects and advantages of the present invention shall become apparent from the detailed description and figures provided herewith.

### BRIEF DESCRIPTION OF THE FIGURES

FIG. 1 is a side view of one embodiment of a bone screw.

FIG. 2 is a schematic side view illustrating the external profile of the bone screw of FIG. 1.

FIG. 3 is a perspective view of the bone screw of FIG. 1 engaged with an interbody spacer.

FIG. 4 is a section view of an alternative embodiment of a bone screw.

FIG. 5 is a side view of another alternative embodiment of a bone screw.

FIG. 6 is a side view of another alternative embodiment of a bone screw.

FIG. 7 is a side view of another alternative embodiment of a bone screw.

FIG. 8 is a side view of another alternative embodiment of a bone screw.

FIG. 9 is a side view of another alternative embodiment of a bone screw.

FIG. 10 is a side view of another alternative embodiment of a bone screw.

FIG. 11 is a side view of another alternative embodiment of a bone screw.

FIG. 12 is a side view of another alternative embodiment of a bone screw.

FIG. 13 is a side view of another alternative embodiment of a bone screw.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is hereby intended, and that alterations and further modifications to the illustrated devices and/or further applications of the principles of the invention as illustrated herein are contemplated as would normally occur to one skilled in the art to which the invention relates.

The present invention is generally directed to a variety of unique bone screws which, in one form, may be used to engage with and attach an implant to bone or bony tissue, although it should be appreciated that alternative uses for the bone screws described herein are contemplated. In one form, the bone screws are configured to be engaged within a passage of an implant such that an interference fit is provided between the bone screw and the implant. More particularly, the sidewall of the implant passage or the external surface of the bone screw is provided with a surface hardness that is greater than the other, and one or both of the passage and the bone screw include a geometrical configuration that causes the sidewall of the passage or the bone screw, whichever has the lower surface hardness, to become deformed. This deformation results in the interference fit between the bone screw and the implant, and thereby reduces the likelihood of the bone screw from becoming disengaged from the implant and/or the bone.

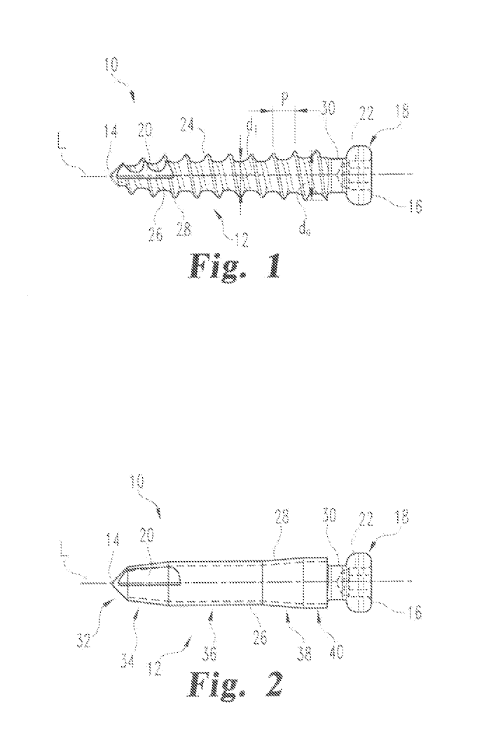

Referring now to FIG. 1, shown therein is a bone screw 10 according to one embodiment of the present invention. The bone screw 10 extends along a longitudinal axis L and includes an elongate shank portion 12 that extends between a distal tip 14 and a proximal end portion 16 that includes a head portion 18. The bone screw 10 may be formed of any suitable biocompatible material including but not limited to titanium, titanium alloy, stainless steel, metallic alloys, polyaryletherketone (PAEK), polyetheretherketone (PEEK), carbon-reinforced PEEK, polyetherketoneketone (PEKK), polysulfone, polyetherimide, polyimide, ultra-high molecular weight polyethylene (UHMWPE), and plastics, just to name a few possibilities.

In one embodiment, the distal tip 14 of the elongate shank portion 12 is configured to penetrate bone. For example, in the illustrated embodiment, the distal tip 14 is tapered or pointed to facilitate entry into bone. However, in other embodiments, the distal tip 14 may define a blunt or rounded end. The elongate shank portion 12 also includes a cutting flute 20 extending proximally from the distal tip 14 to provide the bone screw 10 with self-cutting or self-tapping capabilities, although other embodiments were the cutting flute 20 is not provided are also contemplated. In still other embodiments, the bone screw 10 may be provided with an axial passage (not shown) extending from the proximal end portion 16 and partially or entirely therethrough to define a cannulation opening, and may be further provided with transverse passages that communicate with the axial passage to define fenestration openings. The cannulation and fenestration openings may be used to deliver material such as, for example, bone cement from the proximal end portion 16 of the bone screw 10 and into areas of the bone axially or laterally adjacent the distal tip 14 or other portions of the elongate shank portion 12.

In the illustrated embodiment, the head portion 18 is enlarged relative to the elongate shank portion 12 and is generally structured to bear against an implant, although other configurations of the head portion 18 are contemplated. For example, in one non-illustrated form, the head portion 18 may be the same size as or smaller than the elongate shank portion 12. The bone screw 10, and particularly the head portion 18, preferably includes features that allow for releasable engagement with a driving tool or instrument (not shown) such as, for example, a screwdriver. More particularly, in the illustrated form, the head portion 18 includes a receptacle 22 that is sized and shaped to receive a distal end portion of a driver tool. The receptacle 22 may be non-circular such as, for example, hexagonal or rectangular shaped to provide non-rotational engagement between the head portion 18 and the driver tool to facilitate driving engagement of the bone screw 10 into bone. More particular examples of non-circular configurations for the receptacle 22 include, but are not limited to, slotted, Phillips, hexagonal, Torx, spline drive, and double hex configurations. Alternatively, the head portion 18 may define external surface features configured for engagement by the distal end portion of a driver tool.

The bone screw 10 also includes a threading 24 that extends along the elongate shank portion 12 from the distal tip 14 toward the proximal end portion 16. In the illustrated embodiment, the threading 24 terminates distally of the head portion 18. Similarly, the elongate shank portion 12 includes a non-threaded, reduced diameter neck portion 30 positioned between the threading 20 and the head portion 18. However, in other non-illustrated forms, it is contemplated that the threading 24 may continue up to the head portion 18 and/or extend around all or a portion of the head portion 18 to the proximal end portion 16. The threading 24 includes a single thread lead in the form of a helical thread pattern that wraps around the elongate shank portion 12 along the longitudinal axis L. The threading 24 defines a thread root 26 in proximity to the longitudinal axis L, and a thread crest 28 distanced from the longitudinal axis L and radially spaced from the thread root 26. The elongate shank portion 12 includes an inner thread root diameter dᵢ measured across the thread roots 26, and an outer thread diameter dₒ measured across the thread crests 28. In one form, the thread crests 28 may be tapered to facilitate self-tapping, or may be truncated and substantially flat. It should further be appreciated that the profile of the thread crests may be the same, or may vary along the length of the elongate shank portion 12.

In the illustrated embodiment, the depth of the threading 24 between the thread root 26 and the thread crest 28 is constant and uniform along the length of the elongate shank portion 12. However, in alternative embodiments, it is contemplated that the depth of the threading 24 may vary along one or more portions of the elongate shank portion 12. In addition, the pitch P of the threading 24 is constant and uniform along the length of the elongate shank portion 12, although in alternative embodiments it is contemplated that the pitch P of the threading 24 may vary along one or more portions of the elongate shank portion 12.

While not previously discussed, it should be appreciated that the external profile of the elongate shank portion 12 may vary along its length. More particularly, FIG. 2 provides a schematic, side view of the bone screw 10 which illustrates the variations in the external profile of the elongate shank portion 12 along its length. In FIG. 2, the dashed lines extending along the elongate shank portion 12 illustrate the inner thread root diameter dᵢ defined by the thread roots 26, and the solid lines illustrate the outer thread diameter dₒ defined by the thread crests 28, or the external surface of the bone screw 10 in those portions where the threading 24 is not present. More particularly, in those portions of the elongate shank portion 12 where the threading 24 is present, the outer thread diameter dₒ defines the external profile of that portion of the elongate shank portion 12. Similarly, in those portions of the elongate shank portion 12 where the threading 24 is not present, the external surface of the elongate shank portion 12 defines its external profile.

As illustrated in FIG. 2, the elongate shank portion 12 generally includes a first portion 32, a second portion 34, a third portion 36, a fourth portion 38 and a fifth portion 40 that have different external profiles. For example, with respect to the first portion 32, it is generally defined by the distal tip 14 and includes a tapered or pointed external profile that corresponds to the external surface of the distal tip 14 and widens toward the proximal end portion 16. In alternative forms, it is contemplated that the threading 24 may extend along at least a portion of the distal tip 14 such that the external profile of the first portion 32 is at least partially defined by the corresponding outer thread diameter dₒ of the respective portion of the threading 24. The second portion 34 is positioned proximally of the first portion 32, and includes an external profile having a tapered shape that increases toward the proximal end portion 16. Similarly, it should be appreciated that the outer thread diameter dₒ of the threading 24 along the second portion 34 increases toward the proximal end portion 16. The second portion 34 terminates adjacent to or merges into the third portion 36, which has an external profile having a linear shape. More particularly, the outer thread diameter dₒ of the threading 24 along the third portion 36 is constant and uniform, and generally corresponds to the largest outer thread diameter dₒ of the threading 24 along the second portion 34. However, in other forms, it is contemplated that the outer thread diameter dₒ of the threading 24 along the third portion 36 may be larger or smaller than the largest outer thread diameter dₒ of the threading 24 along the second portion 34.

The fourth portion 38 is positioned proximally of the third portion 36 and includes an external profile having a tapered shape that increases toward the proximal end portion 16. Similarly, it should be appreciated that the outer thread diameter dₒ of the threading 24 along the fourth portion 38 increases toward the proximal end portion 16. The fourth portion 38 terminates adjacent to or merges into the fifth portion 40 which has an external profile having a linear shape. More particularly, the outer thread diameter dₒ of the threading 24 along the fifth portion 40 is constant, and generally corresponds to the largest outer thread diameter dₒ of the threading 24 along the fourth portion 38. However, in other forms, it is contemplated that the outer thread diameter dₒ of the threading 24 along the fifth portion 40 may be larger or smaller than the largest outer thread diameter dₒ of the threading 24 along the fourth portion 38. In the illustrated form, the threading 24 terminates at the proximal end of the fifth portion 40. In addition, the neck portion 30 is positioned between the fifth portion 40 and the proximal head 18 and includes a linear external profile having a reduced diameter relative to the external profile of the fifth portion 40.

In the illustrated embodiment, the external profiles of the second portion 34 and the fourth portion 38 are generally defined by a linear taper that widens toward the proximal end portion 16. However, in one or more alternative forms, it is contemplated that the external profiles of the second portion 34 and the fourth portion 38 may be defined by a curved taper. In addition, in the illustrated embodiment, the taper rate of the external profile of the second portion 34 is more gradual than the taper rate of the external profile of the fourth portion 38. However, in alternative forms, the tapers along the external profiles of the second portion 34 and the fourth portion 38 may be the same, or the taper rate of the external profile of the fourth portion 38 may be more gradual than the taper rate of the external profile of the second portion 34. Moreover, while not previously discussed, it should be appreciated that the difference between the inner thread root diameter dᵢ and the outer thread diameter dₒ along the elongate shank portion 12 is generally constant, although other forms are also contemplated wherein the difference between the inner thread root diameter dᵢ and the outer thread diameter dₒ changes along one or more portions of the elongate shank portion 12. Stated alternatively, in FIG. 2 the dashed lines that illustrate the inner thread root diameter dᵢ and the solid lines that illustrate the outer thread diameter dₒ extend generally parallel to one another, thereby indicating that the depth of the threading 24 is constant along the elongate shank portion 12. However, in one or more alternative forms, it is contemplated that only one of the inner thread root diameter dᵢ and the outer thread diameter dₒ is provided with a taper along certain portions of the elongate shank portion 12, or that the inner thread root diameter dᵢ and the outer thread diameter dₒ along elongate shank portion 12 may be provided with tapers that are different from each other.

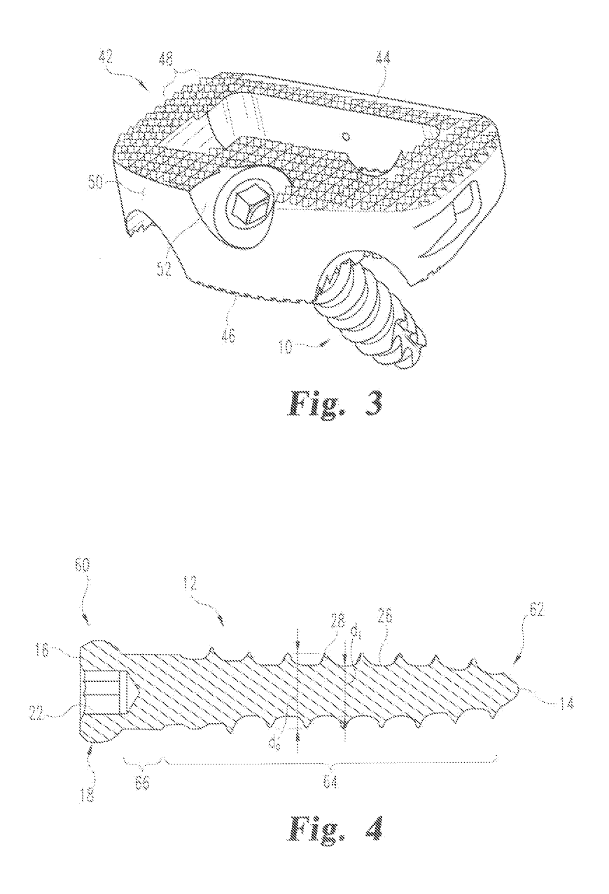

With reference to FIG. 3, the bone screw 10 is engaged to an implant 42 which in the illustrated form is a spacer configured to be positioned within an intervertebral space between adjacent vertebrae. However, it should be appreciated that, among other alternatives, the bone screw 10 may also be used with an artificial disk or a bone plate, just to name a few possibilities. The implant 42 includes a superior surface 44 configured to engage a first vertebral body, and an inferior surface 46 configured to engage a second vertebral body. Teeth 48 may extend outward from one or both of the surfaces 44, 46 to facilitate insertion into the intervertebral space and/or to maintain the position of the implant 42 within the intervertebral space. A sidewall 50 extends laterally around the implant 42 between the surfaces 44, 46. The implant 42 also includes a passage 52 extending through a portion of the superior surface 44 and the sidewall 50.

The passage 52 is generally configured to receive and engage with the bone screw 10 such that an interference fit between the bone screw 10 and the implant 42 is provided. For example, in the illustrated form, the fourth portion 38 and the fifth portion 40 of the elongate shank portion 12 generally provide an external configuration that creates an interference fit between the bone screw 10 and the implant 42 as the bone screw 10 is axially inserted into the passage 52. For example, the fourth portion 38 and/or the fifth portion 40 may be provided with an external diameter that is greater than the internal diameter of the passage 52 of the implant 42. Similarly, since the passage 52 and/or the implant 42 are constructed of a material that has a different hardness than the bone screw 10 (see discussion above), engagement of the distal portion of the fourth portion 38 with the passage 52 of the implant 42 may begin deformation of one of the bone screw 10 and the passage 52. This deformation continues until the bone screw 10 is seated within the passage 52 of the implant 42, and ultimately results in an interference fit between the bone screw 10 and the implant 42 that reduces the likelihood that the bone screw 10 will become disengaged from the implant 42. Further details regarding the relationship, including the interference fit, between the bone screw 10 and the implant 42, as well as between other bone screws and implants, are provided in commonly-owned U.S. patent application Ser. No. 12/508,669, the contents of which are incorporated herein by reference in their entirety.

Other alternative embodiment bone screws are also contemplated for providing an interference fit with an implant. For example, one alternative embodiment bone screw 60 is illustrated in cross section view in FIG. 4, with like reference numerals referring to like features of the bone screw 10 previously described above. The elongate shank portion 12 of the bone screw 60 generally includes a first portion 62, a second portion 64 and a third portion 66 that each have different external profiles. More particularly, with respect to the first portion 62, it is generally defined by the distal tip 14 and includes a tapered or pointed external profile that corresponds to the external surface of the distal tip 14 and widens toward the proximal end portion 16. In alternative forms, it is also contemplated that the threading 24 may extend along at least a portion of the distal tip 14 such that the external profile of the first portion 62 is at least partially defined by the corresponding outer thread diameter dₒ of the respective portion of the threading 24. The second portion 64 is positioned proximally of the first portion 62, and includes an external profile having a tapered shape that increases toward the proximal end portion 16. Similarly, it should be appreciated that the outer thread diameter dₒ of the threading 24 along the second portion 64 increases toward the proximal end portion 16. In addition, the inner thread root diameter dᵢ also increases toward the proximal end portion 16 such that the depth of the threading 24 remains constant along the elongate shank portion 12. However, as discussed above with respect to the bone screw 10, it is contemplated that the depth of the threading 24 could be varied along one or more portions of the elongate shank portion 12.

The second portion 64 and the threading 24 terminate at the distal end of the third portion 66. The third portion 66 is non-threaded and includes an external profile having a linear shape. Moreover, in the illustrated form, the third portion 66 includes a reduced diameter relative to the largest outer thread diameter dₒ of the threading 24 along the second portion 64. In this embodiment, an interference fit between the bone screw 60 and an implant can be provided as the tapered external profile along the second portion 64 and the proximal end of the second portion 64 are advanced through a passage in the implant.

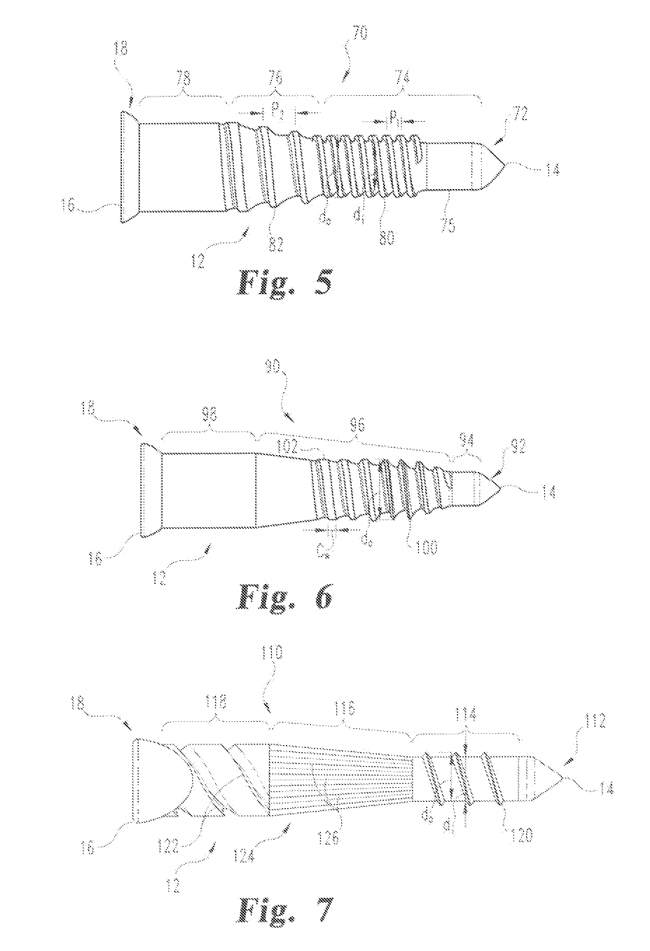

Another alternative embodiment bone screw 70 is illustrated in FIG. 5, where like reference numerals refer to like features of the bone screw 10 previously described above. The elongate shank portion 12 of the bone screw 70 generally includes a first portion 72, a second portion 74, a third portion 76 and a fourth portion 78 that have different external profiles. More particularly, with respect to the first portion 72, it is generally defined by the distal tip 14 and includes a tapered or pointed external profile that corresponds to the external surface of the distal tip 14 and widens toward the proximal end portion 16. The second portion 74 is positioned proximally of the first portion 72, and includes an external profile having a non-tapered, linear shape. The second portion 74 includes a non-threaded section 75 and a threading 80. The threading 80 is positioned adjacent to and proximal of the non-threaded section 75. Moreover, it should be appreciated that the non-tapered, linear external profile of the second portion 74 is defined by both the non-threaded section 75 and the threading 80. Similarly, it should further be appreciated that the outer thread diameter dₒ of the threading 80 along the second portion 74 is constant and uniform.

The second portion 74 terminates at a distal end of the third portion 76. The third portion 76 is positioned proximally of the second portion 74 and includes a threading 82 and an external profile having a tapered shape that increases toward the proximal end portion 16. Similarly, it should be appreciated that the outer thread diameter dₒ of the threading 82 along the third portion 76 increases toward the proximal end portion 16. In addition, the pitch Pᵢ of the threading 80 is different than the pitch P₂ of the threading 82. More particularly, the pitch P₂ of the threading 82 is greater than the pitch Pᵢ of the threading 80. In an alternative embodiment, it is contemplated that the threading 80 and the threading 82 could be replaced by a single threading that includes a continuous helical thread that is provided with different pitches along the second portion 74 and the third portion 76. In the illustrated form, the threading 80 and the threading 82 generally include a constant depth along the second portion 74 and the third portion 76, respectively. However, it should be appreciated that alternative forms in which the depth of the threading 80 and/or the threading 82 changes along one or more portions of the second portion 74 and/or the third portion 76, respectively, are also contemplated. Moreover, in addition to having different pitches, it should be appreciated that the threading 80 and the threading 82 could be provided with other different or varying aspects such as, for example thread angle and crest width, among other possibilities.

The third portion 76 terminates adjacent to or merges into the fourth portion 78 which has an external profile having a linear shape. More particularly, the fourth portion 78 generally has a constant diameter that generally corresponds to the largest outer thread diameter dₒ of the threading 82 along the third portion 76. However, in other forms, it is contemplated that the diameter of the fourth portion 78 may be larger or smaller than the largest outer thread diameter dₒ of the threading 82 along the third portion 76. In the illustrated form, the threading 82 extends partially into the fourth portion 78, such that the fourth portion 78 includes both threaded and non-threaded sections. However, in other forms, it is contemplated that the threading 82 may terminate distally of the fourth portion 78 such that it only has a non-threaded configuration.

Another alternative embodiment bone screw 90 is illustrated in FIG. 6, where like reference numerals refer to like features of the bone screw 10 previously described above. The elongate shank portion 12 of the bone screw 90 generally includes a first portion 92, a second portion 94, a third portion 96 and a fourth portion 98 that have different external profiles. More particularly, with respect to the first portion 92, it is generally defined by the distal tip 14 and includes a tapered or pointed external profile that corresponds to the external surface of the distal tip 14 and widens toward the proximal end portion 16. The second portion 94 is non-threaded and positioned proximally of the first portion 92. The second portion 94 also includes an external profile having a non-tapered, linear shape.

The second portion 94 terminates at a distal end of the third portion 96. The third portion 96 is positioned proximally of the second portion 94 and includes a threading 100 and an external profile having a tapered shape that increases toward the proximal end portion 16. Similarly, it should be appreciated that the outer thread diameter dₒ of at least a portion of the threading 100 along the third portion 96 increases toward the proximal end portion 16. In addition, the threading 100 only extends along a portion of the second portion 96 such that the second portion 96 is provided with a non-threaded portion positioned proximally of the threading 100. Thus, it should be appreciated that the tapered external profile of the third portion 96 is defined by both a non-threaded section and the threading 100. Moreover, in the illustrated form, the crest width c_{w} of the crests 102 of the threading 100 increases toward the proximal end portion 16. However, in another form, it is contemplated that another aspect of the threading 100 could change along the third portion 96.

The third portion 96 terminates adjacent to or merges into the fourth portion 98 which is non-threaded and has an external profile having a linear shape. More particularly, the fourth portion 98 generally has a constant diameter that corresponds to the diameter of the third portion 96 at its proximal end adjacent to the fourth portion 98. However, in other forms, it is contemplated that the diameter of the fourth portion 98 may be larger or smaller than the diameter of the third portion 96 at its proximal end.

Another alternative embodiment bone screw 110 is illustrated in FIG. 7, where like reference numerals refer to like features of the bone screw 10 previously described above. The elongate shank portion 12 of the bone screw 110 generally includes a first portion 112, a second portion 114, a third portion 116 and a fourth portion 118 that have different external profiles. More particularly, with respect to the first portion 112, it is generally defined by the distal tip 14 and includes a tapered or pointed external profile that corresponds to the external surface of the distal tip 14 and widens toward the proximal end portion 16. The second portion 114 is positioned proximally of the first portion 112, and includes an external profile having a non-tapered, linear shape. The second portion 114 includes a threading 120 that is adapted for engagement with bone and has a constant outer thread diameter dₒ. However, in other forms it is contemplated that the threading 120 could have a tapered outer thread diameter dₒ that increases toward the proximal end portion 16. It is also contemplated that certain aspects of the threading 120, such as the crest width, depth, or pitch for example, could change or vary along the second portion 114. In the illustrated form, the threading 120 terminates distally of the third portion 116.

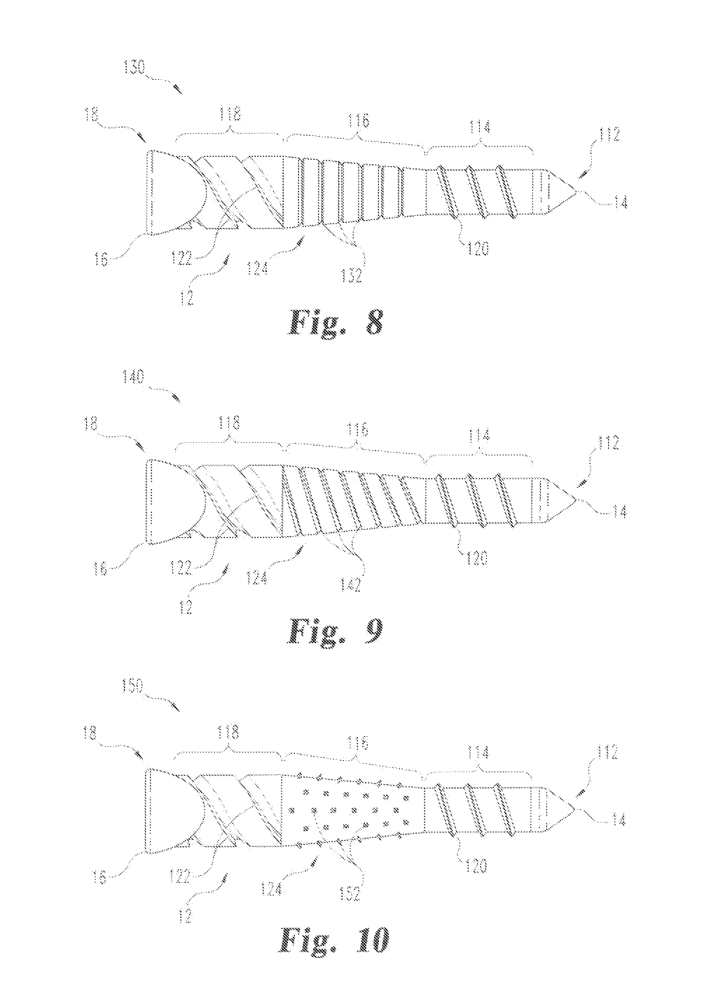

The second portion 114 terminates at a distal end of the third portion 116. The third portion 116 is positioned proximally of the second portion 114 and includes an external profile having a tapered shape that increases toward the proximal end portion 16. The third portion 114 is non-threaded and includes a plurality of engagement features 124 positioned thereon that are adapted to engage with an internal passage of an implant. More particularly, as illustrated in FIG. 7, the engagement features 124 are defined by a plurality of elongate grooves 126 that have groove widths that widen toward the proximal end portion 116. Still, in other embodiments, it is contemplated that alternative engagement features 124 could be provided on the third portion 116. For example, in FIGS. 8-10, where like reference numerals refer to like features of the bone screw 10 and the bone screw 110 described herein, bone screws 130, 140, and 150 each have different engagement features 124 positioned on the third portion 116 of the elongate shank portion 12. More particularly, the engagement features 124 positioned on the third portion 116 of the elongate shank portion 12 of the bone screw 130 are defined by a plurality of circumferential grooves 132 that extend radially about the longitudinal axis of the bone screw 130. With respect the bone screw 140, the engagement features 124 positioned on the third portion 116 of the elongate shank portion 112 are defined by a helical groove 142 extending helically about the longitudinal axis of the bone screw 140. Moreover, the engagement features 124 positioned on the third portion 116 of the elongate shank portion 112 of the bone screw 150 are defined by a surface roughening or surface projections 152, non-limiting examples of which include knurling or spikes, respectively.

The third portion 116 terminates adjacent to or merges into the fourth portion 118 which has an external profile having a linear shape. More particularly, the fourth portion 118 generally has a constant diameter that corresponds to the diameter of the third portion 116 at its proximal end adjacent to the fourth portion 118. However, in other forms, it is contemplated that the diameter of the fourth portion 118 may be larger or smaller than the diameter of the third portion 116 at its proximal end. The fourth portion 118 also includes a helical cutting flute 122 that extends from adjacent to the proximal end of the third portion 116 toward the proximal end portion 16. In a non-illustrated form, it is contemplated that the cutting flute 122 could also extend along at least a portion of the third portion 116. Moreover, in other non-illustrated forms, it is contemplated that the fourth portion 118 could be provided with more than one cutting flute 122, or that the cutting flute 122 could have a non-helical configuration such as, for example, a cutting flute that extends longitudinally along the fourth portion 118. In addition, in one or more forms, it is contemplated that the cutting flute 122 may not be provided on the fourth portion of the bone screws 110, 130, 140 and 150.

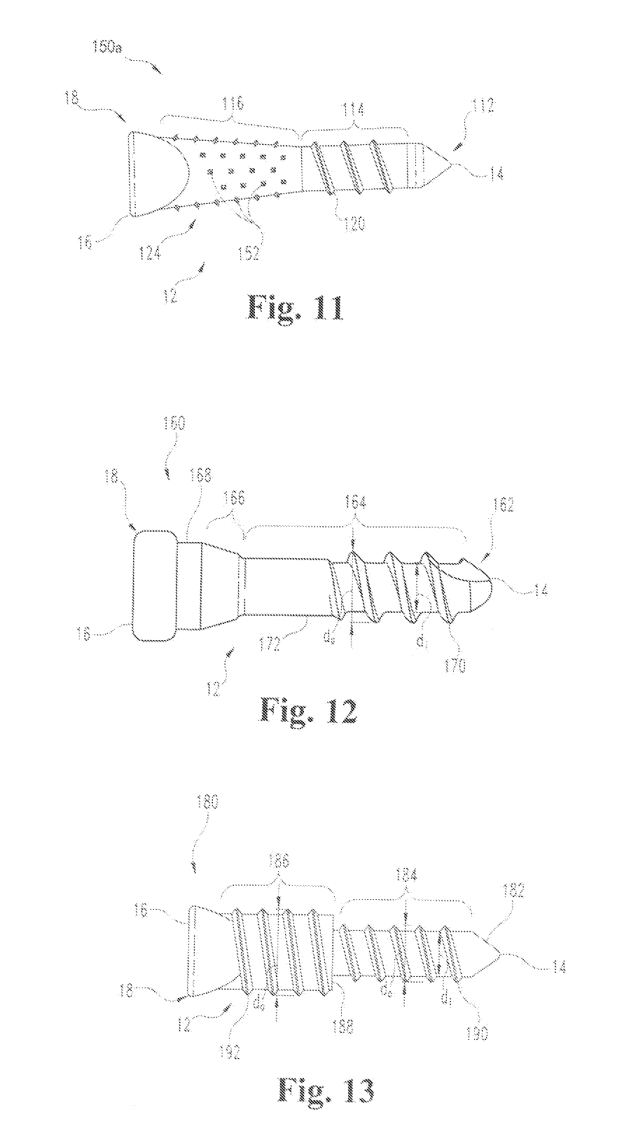

Another alternative embodiment bone screw 150a is illustrated in FIG. 11, where like reference numerals refer to like features of the bone screw 10 previously described above. The bone screw 150a is substantially similar to the bone screw 150, although the elongate shank portion 12 of the bone screw 150a does not include fourth portion 118. Similarly, the third portion 116 of the elongate shank portion 12 of the bone screw 150a extends to the proximal end portion 16. Moreover, while the third portion 116 of the bone screw 150a includes engagement features 124 in the form of surface projections 152, it should be appreciated that in alternative forms the engagement features 124 could be provided in any of the other forms discussed above with respect to the bone screws 110, 130 and 140. In addition, it should be appreciated that the bone screw 150a can be sized such that it generally corresponds in size to the bone screws 110, 130, 140 and 150, although the bone screw 150a may also be sized differently than the bone screws 110, 130, 140 and 150.

Another alternative embodiment bone screw 160 is illustrated in FIG. 12, where like reference numerals refer to like features of the bone screw 10 previously described above. The elongate shank portion 12 of the bone screw 160 generally includes a first portion 162, a second portion 164, a third portion 166 and a fourth portion 168 that have different external profiles. More particularly, with respect to the first portion 162, it is generally defined by the distal tip 14 and includes a tapered or pointed external profile that corresponds to the external surface of the distal tip 14 and widens toward the proximal end portion 16. The second portion 164 is positioned proximally of the first portion 162, and includes an external profile having a non-tapered, linear shape. The second portion 164 includes a non-threaded section 172 and a threading 170. The threading 170 is positioned adjacent to and distal of the non-threaded section 172. Moreover, it should be appreciated that the non-tapered, linear external profile of the second portion 164 is defined by both the non-threaded section 172 and the threading 170. Similarly, it should further be appreciated that the outer thread diameter dₒ of the threading 170 along the second portion 164 is constant. Moreover, in the illustrated form, the outer thread diameter dₒ of the threading 170 is slightly larger than the diameter of the non-threaded section 172, although embodiments where the diameter of the non-threaded section 172 is the same as or greater than the outer thread diameter dₒ of the threading 170 are also contemplated.

The second portion 164 terminates at a distal end of the third portion 166. The third portion 166 is non-threaded and is positioned proximally of the second portion 164. The third portion 166 also includes an external profile having a tapered shape that increases toward the proximal end portion 16. The third portion 166 terminates adjacent to or merges into the fourth portion 168 which has an external profile having a linear shape. More particularly, the fourth portion 168 generally has a constant diameter that corresponds to the diameter of the third portion 166 at its proximal end adjacent to the fourth portion 168. However, in other forms, it is contemplated that the diameter of the fourth portion 168 may be larger or smaller than the diameter of the third portion 166 at its proximal end.

In this embodiment, it is contemplated that the threading 170 and the third portion 166 can both be configured to provide an interference fit with an implant as the bone screw 160 is advanced through a passage of the implant. For example, the threading 170 can be provided with an outer thread diameter dₒ that is sized to initially modify or be modified by the passage of the implant. Additionally, the third portion 166 can be sized such that it also modifies or is modified by the passage of the implant as the bone screw 160 is advanced therethrough.

Another alternative embodiment bone screw 180 is illustrated in FIG. 13, where like reference numerals refer to like features of the bone screw 10 previously described above. The elongate shank portion 12 of the bone screw 180 generally includes a first portion 182, a second portion 184, and a third portion 186 that have different external profiles. More particularly, with respect to the first portion 182, it is generally defined by the distal tip 14 and includes a tapered or pointed external profile that corresponds to the external surface of the distal tip 14 and widens toward the proximal end portion 16. The second portion 184 is positioned proximally of the first portion 182, and includes an external profile having a non-tapered, linear shape, although other shapes for the external profile of the first portion 182 are also contemplated. The second portion 184 includes a threading 190 that is adapted for engagement with bone and has a constant outer thread diameter dₒ and a constant inner thread root diameter dᵢ. However, in alternative embodiments, it is contemplated that one or both of the outer thread diameter dₒ and the inner thread root diameter dᵢ of the threading 190 may be varied along the length of the second portion 184.

The second portion 184 terminates at a distal end of the third portion 186. The third portion 186 includes an external profile having a non-tapered, linear shape, although other shapes for the external profile of the third portion 186 are contemplated. The third portion 186 also includes a threading 192 that can be provided with a self tapping leading thread positioned adjacent to the proximal end of the second portion 184. The threading 192 also has a constant outer thread diameter dₒ and a constant inner thread root diameter dᵢ. However, in alternative embodiments, it is contemplated that one or both of the outer thread diameter dₒ and the inner thread root diameter dᵢ of the threading 192 may be varied along the length of the third portion 186. The outer thread diameter dₒ and inner thread root diameter dᵢ of the threading 192 are generally larger than the outer thread diameter dₒ and the inner thread root diameter dᵢ, respectively, of the threading 190. Similarly, the third portion 186 defines an annular surface or shoulder 188 which faces the second portion 184 and, while not-illustrated, could be provided with a chamfered radial edge. In the illustrated embodiment, the shoulder 188 extends radially beyond the threading 190 of the second portion 184. Stated alternatively, the inner thread root diameter dᵢ of the threading 192 is larger than the outer thread diameter dₒ of the threading 190. Moreover, in the illustrated form, the threading 190 and the threading 192 are provided with different thread profiles, although it is also contemplated that the threading 190, 192 could have identical thread profiles. More particularly, the threading 190 has a greater lead than the threading 192. However, it is also contemplated that additional aspects, such as pitch, depth, crest width or thread angle, could be varied between the threading 190 and the threading 192.

It is contemplated that, among other alternatives, the bone screw 180 can be used with an implant that has a passage provided with a tapered portion that widens toward the end at which the bone screw 180 is initially advanced. Thus, as the bone screw 180 is continually advanced into the passage, the annular shoulder 188 and the threading 192 of the third portion 186 engage with the tapered portion of the passage and modify, or are modified by, the passage to provide an interference fit between the bone screw 180 and the implant.

It should be understood that the bone screws described herein may be anchored within any number of vertebral bodies, including a single vertebral body or two or more vertebral bodies. In one embodiment, the bone screws are anchored within the pedicle region of a vertebral body. However, it should be understood that the bone screws may be anchored to other portions or regions of a vertebral body. It should also be understood that the bone screws described herein may be anchored to a posterior, anterior, lateral, posterolateral or anterolateral aspect of a vertebral body. It should further be understood that the bone screws described herein may be attached to any region of the spinal column, including the cervical, thoracic, or lumbar regions of spinal column. It should likewise be understood that the bone screws described herein may be attached to bone structures other than vertebral bodies, such as, for example, bones associated with the arm or leg.

Any theory, mechanism of operation, proof, or finding stated herein is meant to further enhance understanding of the present invention and is not intended to make the present invention in any way dependent upon such theory, mechanism of operation, proof, or finding. It should be understood that while the use of the word preferable, preferably or preferred in the description above indicates that the feature so described may be more desirable, it nonetheless may not be necessary and embodiments lacking the same may be contemplated as within the scope of the application, that scope being defined by the claims that follow. In reading the claims it is intended that when words such as “a,” “an,” “at least one,” “at least a portion” are used there is no intention to limit the claim to only one item unless specifically stated to the contrary in the claim. Further, when the language “at least a portion” and/or “a portion” is used the item may include a portion and/or the entire item unless specifically stated to the contrary.

While the application has been illustrated and described in detail in the drawings and foregoing description, the same is to be considered as illustrative and not restrictive in character, it being understood that only the selected embodiments have been shown and described and that all changes, modifications and equivalents that come within the spirit of the application as defined herein or by any of the following claims are desired to be protected.

What is claimed is:

1. A bone screw, comprising:
an elongate shank extending along a longitudinal axis from an enlarged proximal head portion to a distal tip, said elongate shank including a first portion having a first non-tapered, linear external profile and a second portion having a first tapered external profile that increases toward said proximal head portion, said second portion being positioned between said first portion and said head portion and terminating distally of said proximal head portion.

2. The bone screw of claim 1, wherein said elongate shank further includes a third portion having a second non-tapered, linear external profile and being positioned between said second portion and said proximal head portion.

3. The bone screw of claim 2, wherein said elongate shank further includes a continuous helical thread extending along said first, second and third portions.

4. The bone screw of claim 2, wherein said second portion terminates at a proximal end including an external diameter that continues along said third portion.

5. The bone screw of claim 1, wherein said elongate shank further includes a third portion having a second tapered external profile that increases toward said proximal head portion, said third portion being positioned between said first portion and said distal tip and terminating at a first proximal end including a first external diameter that continues along said first portion.

6. The bone screw of claim 6, wherein:
said elongate shank further includes a fourth portion having a second non-tapered, linear external profile and being positioned between said second portion and said proximal head portion; and
said second portion terminates at a second proximal end including a second external diameter that continues along said fourth portion.

7. The bone screw of claim 6, wherein said elongate shank further includes a continuous helical thread extending along said first, second, third and fourth portions.

8. The bone screw of claim 1, wherein:
said first portion includes a first threading extending toward said proximal head portion and terminating adjacent said second portion, said first threading having a first thread pitch;
said second portion includes a second threading beginning adjacent said first portion and extending toward said proximal head portion, said second threading having a second thread pitch; and
wherein said first thread pitch is lesser than said second thread pitch.

9. The bone screw of claim 1, wherein said second portion includes a first threading extending toward said proximal head portion and a non-threaded section positioned proximally of said first threading.

10. The bone screw of claim 9, wherein said first threading is continuous and includes a crest width that increases toward said proximal head portion.

11. The bone screw of claim 1, wherein:
said first portion includes a first threading extending toward said proximal head portion and terminating adjacent to said second portion; and
said second portion is non-threaded and includes one or more engagement features on an external surface thereof.

12. The bone screw of claim 11, wherein said one or more engagement features comprise one of a plurality of longitudinal grooves, a plurality of radial grooves, one or more helical grooves, and a plurality of surface spikes.

13. The bone screw of claim 11, wherein said elongate shank further includes one or more cutting flutes positioned proximally of said second portion.

14. The bone screw of claim 1, wherein said first portion includes a first threading extending toward said proximal head portion and a non-threaded section positioned between said first threading and said second portion, and said second portion includes a smooth, non-threaded external surface.

15. A stabilization system, comprising:
a bone screw according to claim 1; and
an implant including at least one passage extending therethrough, said passage being configured to engage with at least said second portion of said elongate shank of said bone screw to provide an interference fit between said bone screw and said implant.

16. The system of claim 15, wherein said implant is an intervertebral spacer.

17. The system of claim 15, wherein said elongate shank of said bone screw further includes:
a third portion having a second tapered external profile that increases toward said head portion, said third portion being positioned between said first portion and said distal tip and terminating at a first proximal end including a first external diameter that continues along said first portion;
a fourth portion having a second non-tapered, linear external profile and being positioned between said second portion and said proximal head portion, said second portion terminating at a second proximal end including a second external diameter that continues along said fourth portion; and
a continuous helical thread extending along said first, second, third and fourth portions.

18. The system of claim 15, wherein:
said first portion of said elongate shank includes a first threading extending toward said proximal head portion and terminating adjacent to said second portion;
said second portion of said elongate shank is non-threaded and includes one or more engagement features on an external surface thereof; and
said elongate shank includes a third portion having a second non-tapered, linear external profile and being positioned between said second portion and said proximal head portion, said third portion including one or more cutting flutes.

19. The system of claim 18, wherein said one or more engagement features comprise one of a plurality of longitudinal grooves, a plurality of radial grooves, one or more helical grooves and a plurality of surface spikes.

20. A bone screw, comprising:
an elongate shank extending along a longitudinal axis from an enlarged proximal head portion to a distal tip, said elongate shank including a first threaded portion and a second portion positioned adjacent to and proximal of said first threaded portion, said second portion defining a distal facing annular shoulder.

21. The bone screw of claim 20, wherein said annular shoulder extends radially beyond an external profile of said first threaded portion.

22. The bone screw of claim 20, wherein said second portion is threaded.

23. The bone screw of claim 22, wherein said second portion includes a self-tapping leading thread that extends from adjacent to said annular shoulder toward said proximal head portion.
